(19) United States
(12) Patent Application Publication
(10) Pub. No.: US 2011/0238080 A1
(43) Pub. Date: Sep. 29, 2011

(54) Robotic Surgical Instrument System

(76) Inventors: Date Ranjit, Pune (IN); Date Jaydeep, Pune (IN); Mihir Desai, La Canada, CA (US)

(21) Appl. No.: 13/069,067

(22) Filed: Mar. 22, 2011

Related U.S. Application Data
(60) Provisional application No. 61/282,740, filed on Mar. 25, 2010.

(51) Int. Cl. A61B 19/00

(52) U.S. Cl. 606/130

(57) ABSTRACT

A robotic surgical instrument system for performing a surgical procedure is envisaged wherein the system is a dual articulated arm configuration robot that enables entry into an operative space via an access port. Surgical arms are inserted into the operative space in a substantially straight line and then articulated inside the operative space. The articulation of the surgical arms by a surgical console is achieved using ‘triangulation’.

### FIELD OF THE INVENTION

This invention relates to robotic surgical instrument systems.

### BACKGROUND

Surgery, typically involves an invasive procedure that requires stitches, involves longer healing time, risk of infection, and requires a patient to be under anesthesia for a longer period of time. Laparoscopic surgery, also referred to as minimally invasive surgery, is a boon that solves most of the aforementioned problems, besides being cosmetically appealing to a patient.

An incision is made in a patient's abdomen and the incision may be retracted using a retractor of the type described, for instance, in United States Patent Application US 2005-009071. An access device is attached to the retractor. The access device has a number of access ports each with an instrument seal to effect a seal around a separate instrument extended through the device. Each instrument seal is separate from the other instrument seals and is spaced apart from the other instrument seals. The instrument seals may be used with various instruments and/or camera/scopes. One such access device is also described in United States Patent Application US2009-0036745.

Robot assisted laparoscopic surgeries are performed with limited physical contact between a surgeon and a patient. The surgeon is remote from the patient, working a few feet from the operating table while seated at a computer console with a three-dimensional view of the operating field.

A main drawback associated with robotic systems known in the art is the need for a plurality of incisions in a patient's body and accordingly a plurality of access ports for insertion of surgical arms of the robotic systems.

There is felt a need to overcome this drawback and provide a robotic surgical instrument system that facilitates insertion of surgical arms using only one access port that requires a single incision in a patient's body.

### SUMMARY OF THE INVENTION

In accordance with the present invention, there is provided a robotic surgical instrument system, the system is characterized by:
a plurality of articulating arms having at least two articulation joints, the articulating arms being adapted to be inserted into an operative space in a substantially straight configuration and further adapted to controllably articulate inside the operative space, with at least three degrees of freedom of movement;
at least one access port adapted to receive the articulating arms; and
controlling means adapted to control the articulation of the articulating arms inside the operative space to perform a surgical procedure.

Typically, the access port is selected from the group consisting of gel ports, puncturable sealed ports and ports with pre-punctured openings.

Preferably, in accordance with one embodiment of the present invention, at least two of the articulating arms are surgical arms adapted to hold tools.

Additionally, at least one of the articulating arms is adapted to hold a vision system selected from the group consisting of a fiber optic scope, an insertable camera system and a separate insertable camera.

In accordance with another embodiment of the present invention, there is provided a robotic surgical instrument system, the system characterized by:
a plurality of articulating arms adapted to be inserted into an operative space in a substantially straight configuration and further adapted to controllably articulate inside the operative space, with at least three degrees of freedom of movement;
at least one access port adapted to receive the articulating arms;
controlling means adapted to control the articulation of the articulating arms inside the operative space to perform a surgical procedure; and
at least one vision system adapted to be inserted into the operative space, the vision system being selected from the group consisting of a fiber optic scope, an insertable camera system and a separate insertable camera.

Typically, in accordance with the present invention, the controlling means comprises:
at least two external articulated mounting robots co-operating with the articulating arms, the mounting robots having six degrees of freedom and adapted to be floor mounted or ceiling mounted; and
a surgical console adapted to provide an interface for the surgical procedure by a surgeon.

Additionally, in accordance with the present invention, the controlling means is adapted to attach tools to or detach tools from the articulating arms.

In accordance with an aspect of the invention, the movement of the surgical arms is achieved by a mechanism comprising cables, pulleys and linkages.

In accordance with the present invention, there is provided a method for a robotic surgical system to access an operative space, the method comprising the following steps:
making an incision in a patient's body;
mounting an access port on the incision;
inserting a plurality of articulating arms into an operative space via the access port in a substantially straight configuration;
controlling said articulating arms inside the operative space to reach a pre-determined operation site by triangulation; and
attaching tools to or detaching tools from the articulating arms.

Preferably, in accordance with the method described herein above, the step of inserting includes a step of inserting at least two articulating arms holding tools and at least one vision system.

### BRIEF DESCRIPTION OF ACCOMPANYING DRAWINGS

The foregoing features of the present invention will become more apparent from the following description and appended claims, taken in conjunction with the accompanying drawings. Understanding that these drawings depict only typical embodiments of the invention and are, therefore, not to be considered limiting its scope, the invention will be described with additional specificity and detail through use of the accompanying drawings in which:

FIG. 1 illustrates an isometric view of a robotic surgical instrument system in accordance with the present invention;

FIG. 2 illustrates the insertion of surgical arms of the system of FIG. 1 through an access port;

FIG. 3 illustrates an isometric view of the movement of tools at the end of the surgical arms of the system of FIG. 1;

FIG. 4 illustrates an end view of the movement of tools in an operative space via an access port;

FIGS. 5 to 10 illustrate the system in accordance with the present invention under various operative configurations;

FIG. 11 is a cross sectional view of a pair of surgical arm mounting robots and associated surgical arms of the system of FIG. 1;

FIG. 12 is an isometric view illustrating details of one surgical arm of the system of FIG. 1;

FIG. 13 is an isometric view of motor mounting, pitch-1 base and pitch-1 axis that form part of a surgical arm of the system of FIG. 1;

FIG. 14 is an exploded view of FIG. 13;

FIG. 15 is an isometric view of pitch 2 link, pitch 2 axis and yaw axis that form part of a surgical arm of the system of FIG. 1;

FIG. 16 is an exploded view of FIG. 15;

FIG. 17 is an isometric view of an arm wrist and yaw assembly that form part of a surgical arm of the system of FIG. 1; and

FIG. 18 is an exploded view of FIG. 17.

### DETAILED DESCRIPTION

It will be readily understood that the components of the present invention, as generally described and illustrated in the accompanying drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout. The illustrated drawings are self explanatory and will be obvious to a person skilled in the art.

The systems known in the art are plagued by drawbacks including a need to provide multiple incisions in the patient's body, risks of infection and lesions and a longer time for healing. In accordance with the present invention, there is provided an ergonomically designed robotic surgical instrument system suitable for use during laparoscopic surgery to facilitate access to an insufflated abdominal cavity while maintaining pneumoperitoneum. The system comprises at least two external surgical arm mounting robots co-operating with an associated surgical arm that holds tools for performing a surgical procedure. Each surgical arm is provided with at least two articulation joints. The surgical arms are inserted into the operative space in a substantially straight configuration and manipulated by a surgical console using triangulation in the operative space. The need for a single incision for a single access port and the method of achieving triangulation within the operative space are the main advantages of the present invention that lead to minimum movement of the system on the surface of the patient's body and minimum invasion, thus overcoming the drawbacks of the prior art.

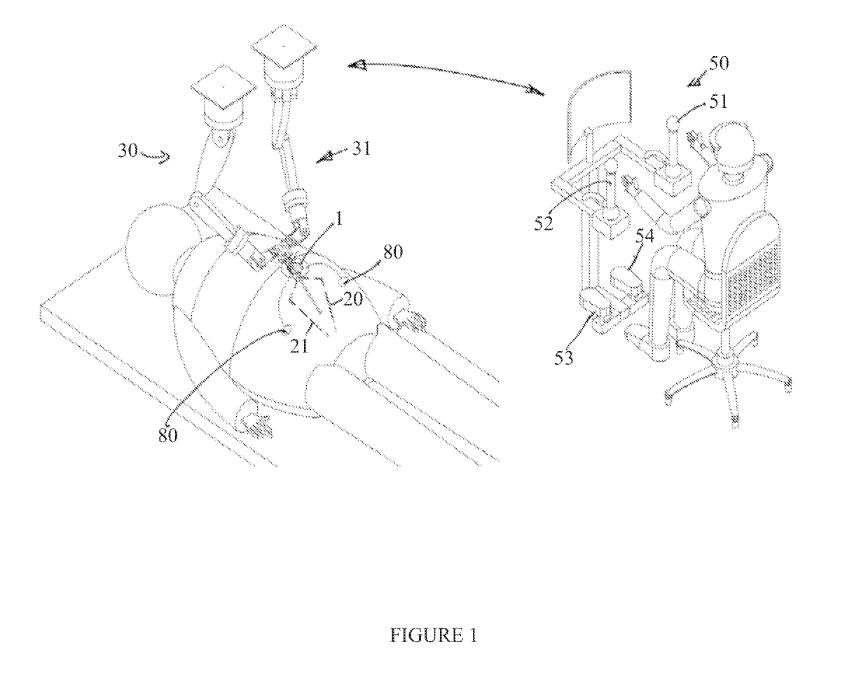

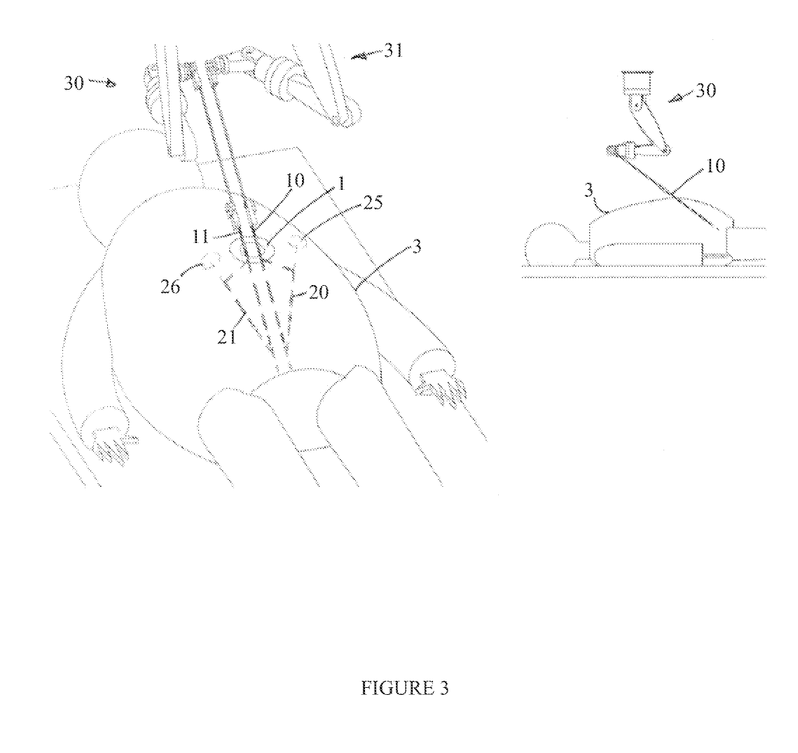

Referring to FIGS. 1 to 3, a robotic surgical instrument system in accordance with the present invention mainly comprises two external surgical arm mounting robots 30,31 and two surgical arms 10, 11 controlled by an external surgical console 50 which typically comprises two hand joysticks 51, 52 and foot controls 53, 54 for manipulation of the surgical arms 10, 11, tools 20, 21, position of the mounting robots 30,31 and a vision system 80.

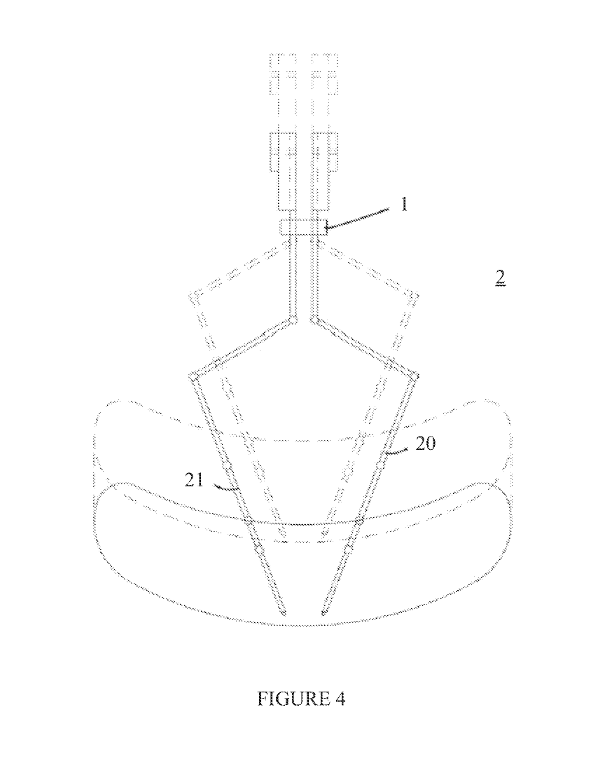

The system in accordance with the present invention is a dual articulated arm configuration robot that enables entry into an operative space 2 in the abdominal cavity via an access port 1 for performing a surgical procedure. The access port is adapted to facilitate unhindered access to the operative space 2. The access port is typically a gel port, a puncturable sealed port or a port with pre-punctured openings. Typically, the access port receives at least two surgical arms 10, 11 and a vision system 80 to be inserted into the operative space 2 via the access port 1. The surgical arms 10, 11 enter the operative space 2 in a substantially straight line, and are then articulated inside the operating space 2 within the patient body, “by triangulation” achieved by the surgical console 50. The process of triangulation typically involves determining a precise operative site by measuring angles to it from known points at either end of a fixed baseline, rather than measuring distances to the site directly. The system in accordance with the present invention enables the advantages of “triangulation” as if operating in a biport configuration. The arms operate as if the tools 20, 21 were inserted in biport configuration through “virtual” ports 25, 26 as per established biport procedures. FIG. 4 illustrates an end view of the movement of the tools 20, 21 in the operative space 2 via the access port 1. The preferred embodiment of the present invention requires a single access port 1 for insertion of the surgical arms 10,11. However, in accordance with an alternative embodiment, the surgical arms are inserted through two discrete access ports.

The two external surgical arm mounting robots 30, 31 are each provided with at least six degrees of freedom for facilitating positioning of the articulated surgical arms 10, 11 with respect to the patient and the bed setup for the surgical procedure.

The two surgical arms 10, 11 are each provided with at least three degrees of freedom that allow the surgical arms 10, 11 to be inserted straight, and then articulate inside the operative space 2, to enable triangulation and micro-motions around the desired operating site.

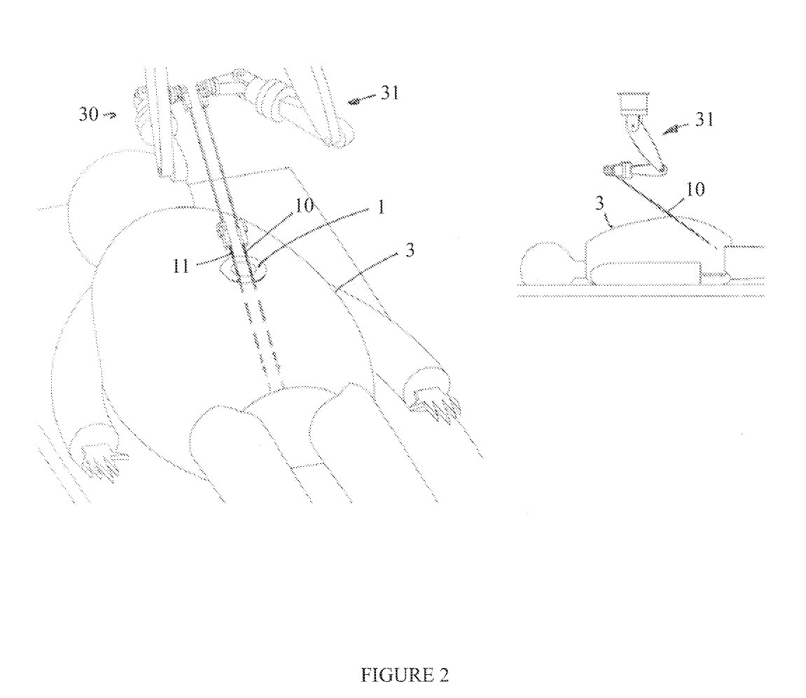

The articulated surgical arm mounting robots 30, 31 enable the X, Y, Z positions and angle of approach to the desired operating site to be achieved in a straight configuration, when surgical arms are inserted as illustrated in FIG. 2. These robots can be floor mounted or ceiling mounted—freeing up the space around the patient for surgeons and assistants.

The system in accordance with the present invention provides a sufficiently large work envelope that enables precision manipulation required for surgical procedures inside the patient's body without significant motion outside the patient's body. This frees up external space, and allows safe operative space for the surgeons/assistants around the robotic system, without keeping a side of the patient occupied by a large moving floor—mounted structure.

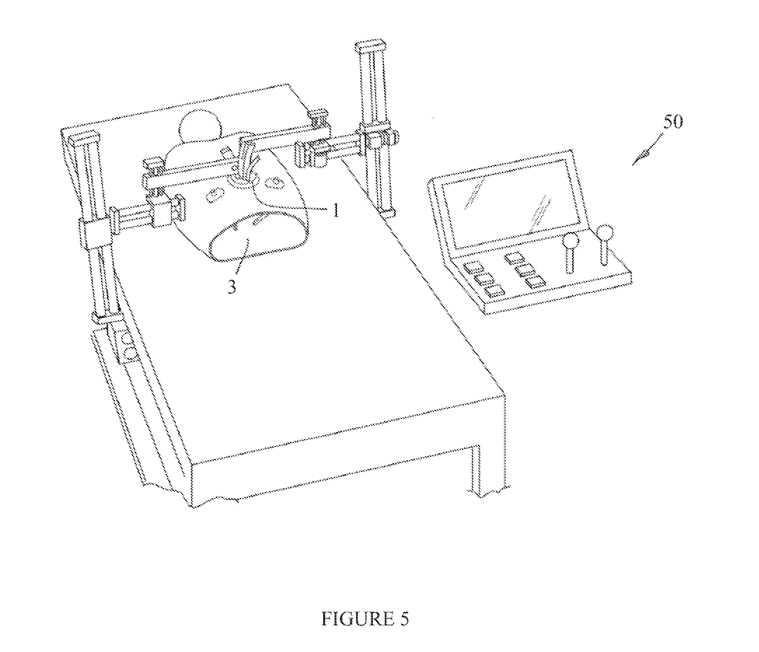

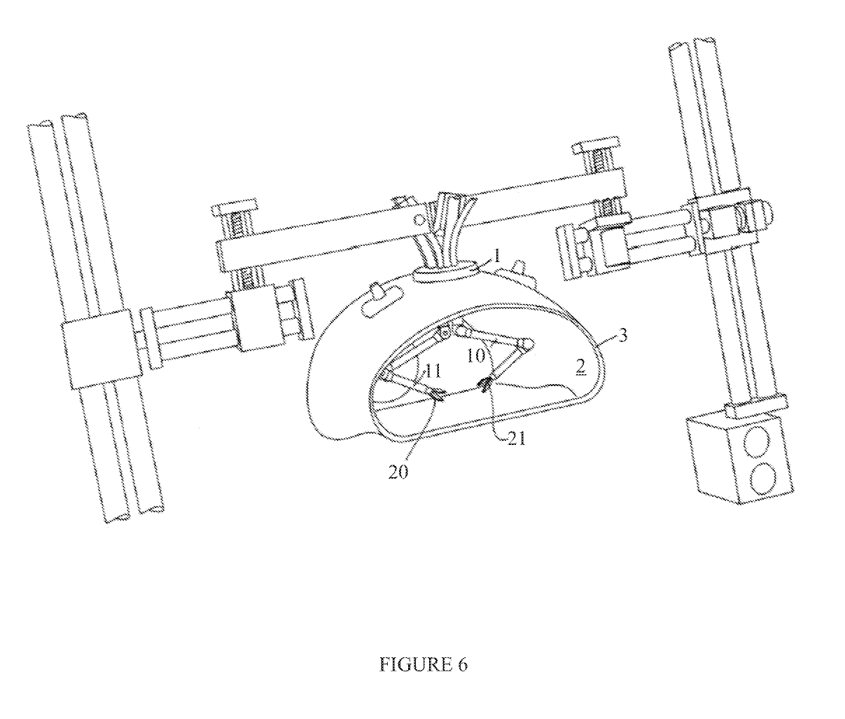

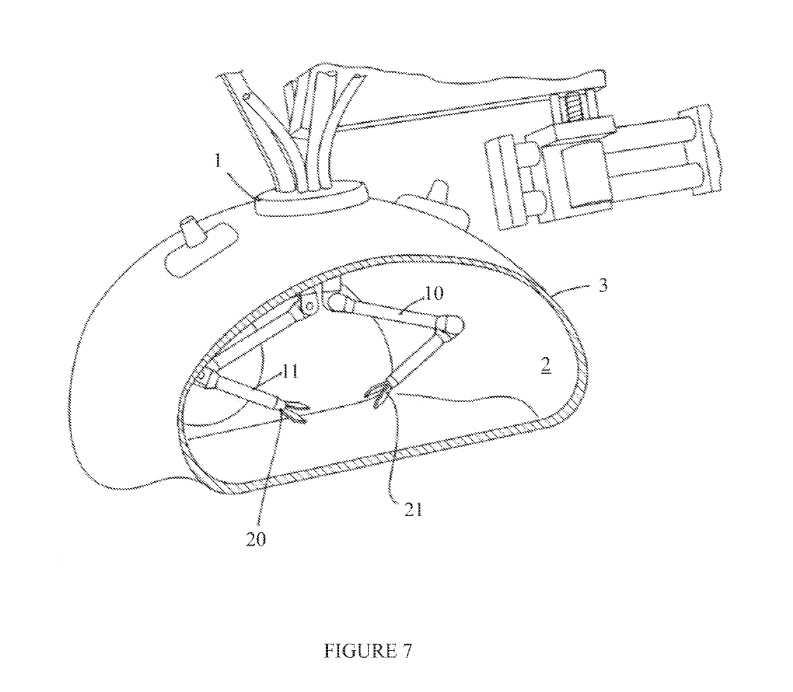

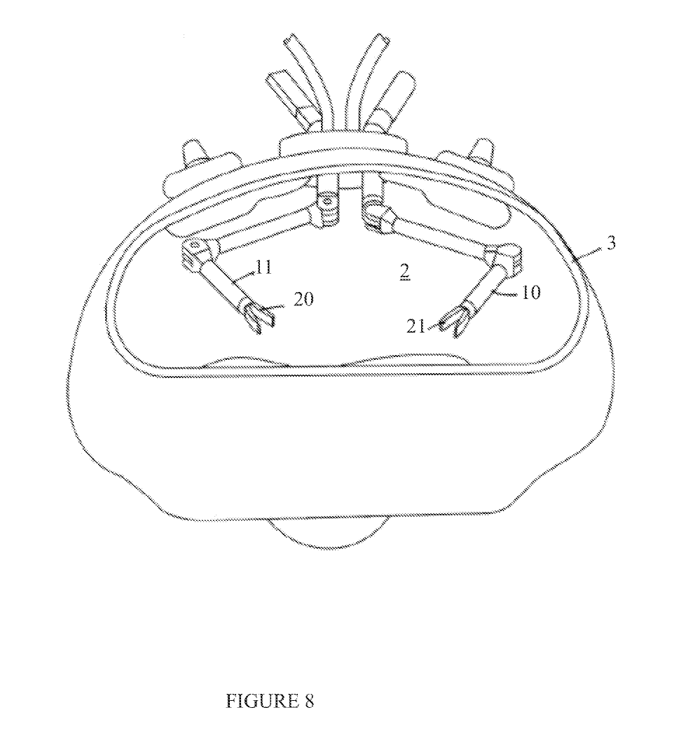

FIGS. 5 to 10 illustrate the system in accordance with the present invention under various operative configurations.

Tools 20, 21 at the end of the surgical arms 10, 11 are attached on or detached from the surgical arms 10, 11 either inside or outside the operative space 2. In one embodiment of the present invention, tools are attached to the surgical arm before insertion of the surgical arm through the access port 1. Alternatively, in accordance with another embodiment, tools are attached to the surgical arm after insertion of the surgical arm through the access port 1. The tool change is performed within the operative space 2 without a requirement to extract the surgical arm fully out, through a separate assistant port (not shown).

The movement of the surgical arms 10, 11 is controlled using a mechanism of cables, pulleys and linkages, configured such that actuation is always achieved by the cables in tension, resulting in precision motion.

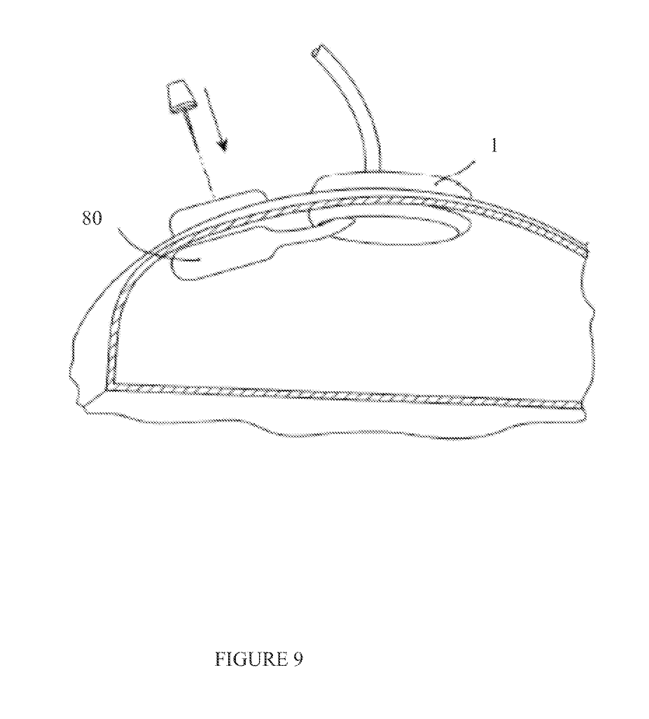

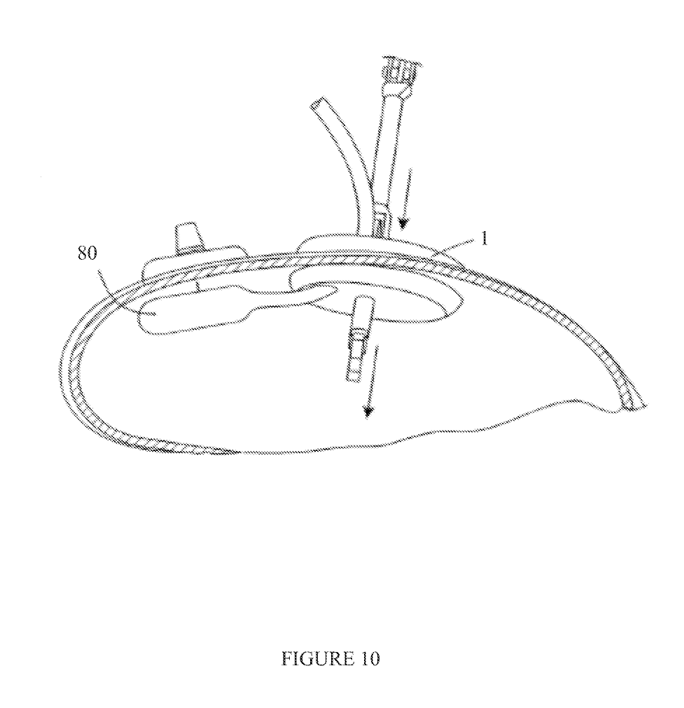

The system in accordance with the present invention further comprises at least one vision system. The vision system is typically a fiber optic scope, an insertable camera system, or a separate insertable camera 80 through an “umbilical chord” cable inserted through the same access port 1 or optionally, another access port (not shown). The camera is anchored to the abdominal wall as illustrated in FIGS. 9 and 10. Preferably, a magnet is used to hold the camera to the abdominal wall. Alternatively, to provide enhanced visibility within the operative space 2, two such cameras 80 or vision systems are provided.

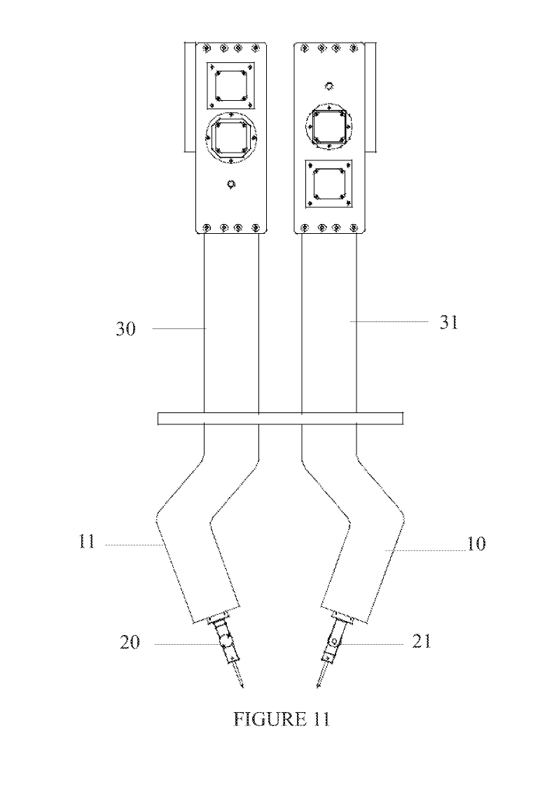

Mechanical details of the construction of the robotic system in accordance with the present invention are illustrated in FIGS. 11 to 18.

Referring to FIG. 11, each of the surgical arm mounting robots 30 and 31 are provided with a motor (not specifically referenced) at each of the articulation joints thereof, wherein each motor facilitates rotation of a pulley which in turn results in tension in the associated cables; the tension in the cables facilitates the movement of the surgical arms 10, 11.

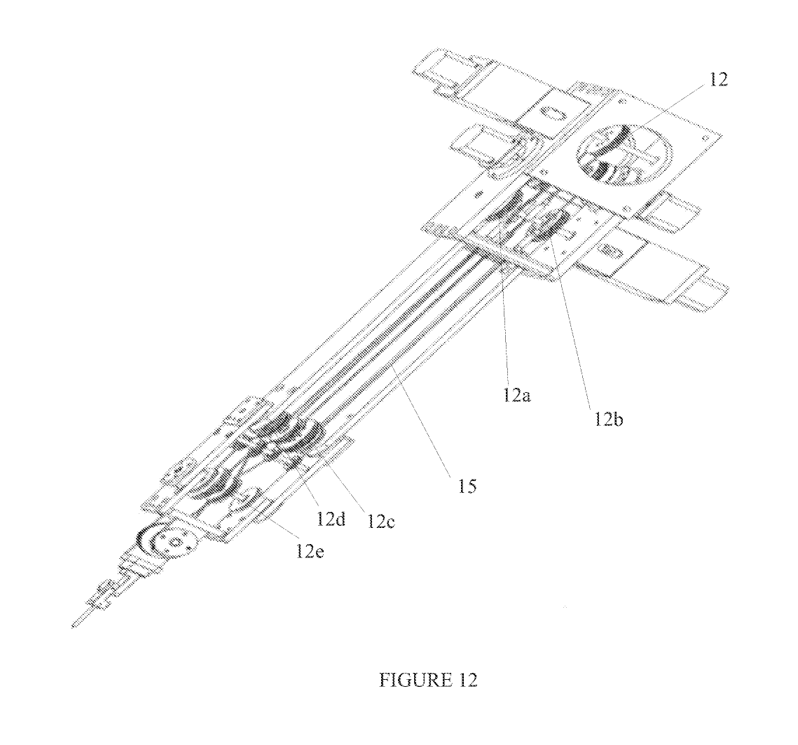

Referring to FIG. 12 of the accompanying drawings, a motor (not specifically referenced) is provided for driving a pulley 12. A cable 15 passes over the pulley 12 and imparts required motion to the surgical arms 10, 11. Further, there are a plurality of idler pulleys 12a-12e provided for tensioning the cable 15 and resulting in precision motion of the surgical arms 10, 11.

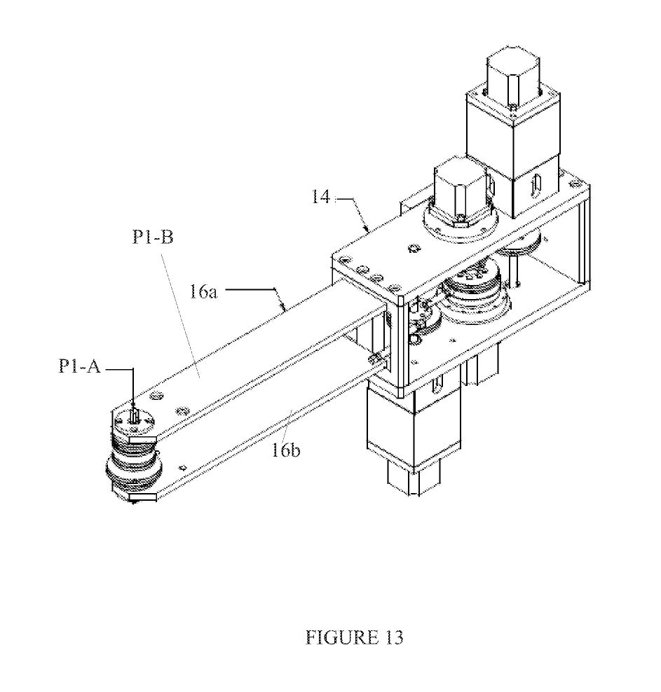

Referring to FIG. 13 of the accompanying drawings, the motor (not specifically referenced) as well as the pulley 12 (shown in FIG. 12) driven by the motor are both housed inside a motor mounting 14. A pitch-1 base P1-B in the form of spaced apart plates 16a and 16b extends outwardly from the motor mounting 14. A pitch-1 axis P1-A is located at a distal end of the pitch-1 base P1-B.

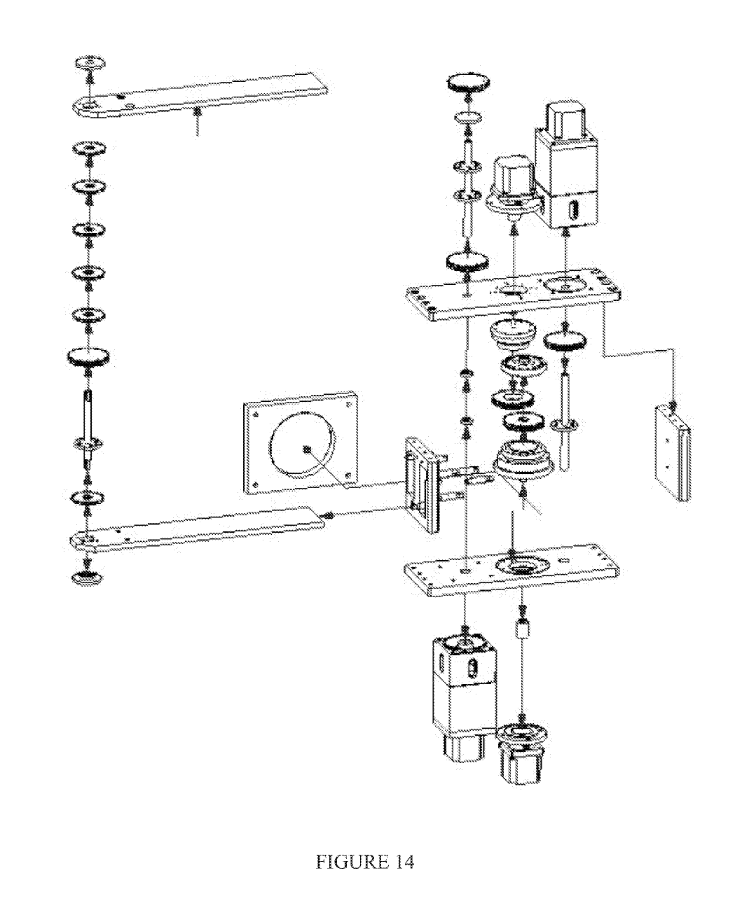

Referring to FIG. 14, the motor mounting 14 comprises a plurality of plates assembled together by a plurality of fastening elements for securely holding the motor and the pulleys there-in.

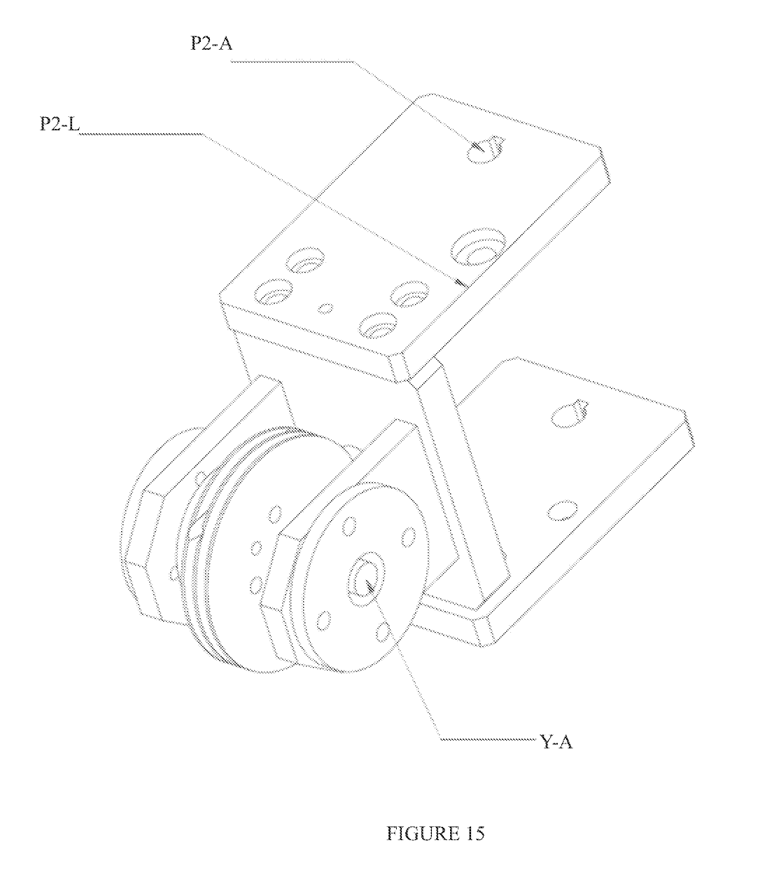

FIG. 15 is an isometric view of a pitch-2 link P2-L, pitch-2 axis P2-A and yaw axis Y-A that form part of a surgical arm of the system of FIG. 1.

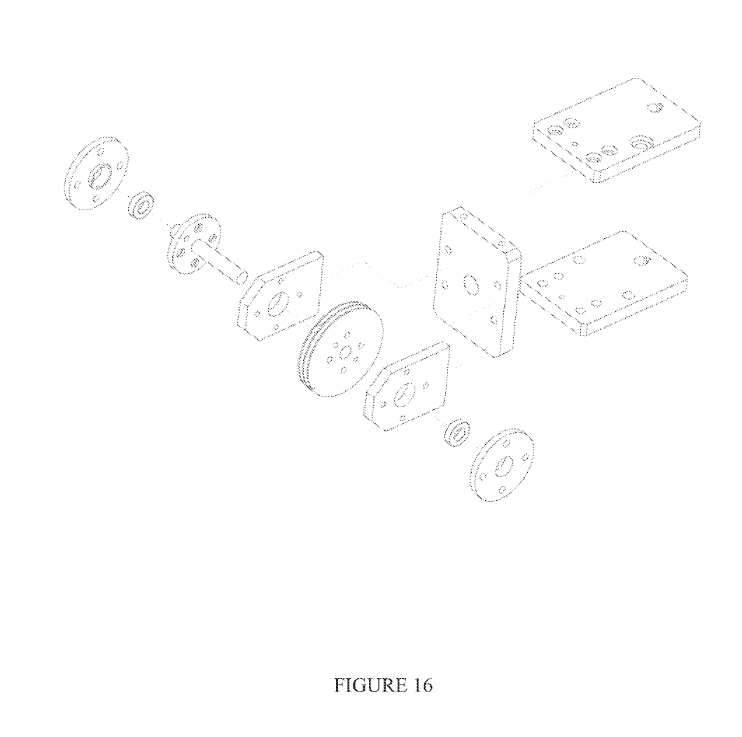

FIG. 16 is an exploded view of FIG. 15.

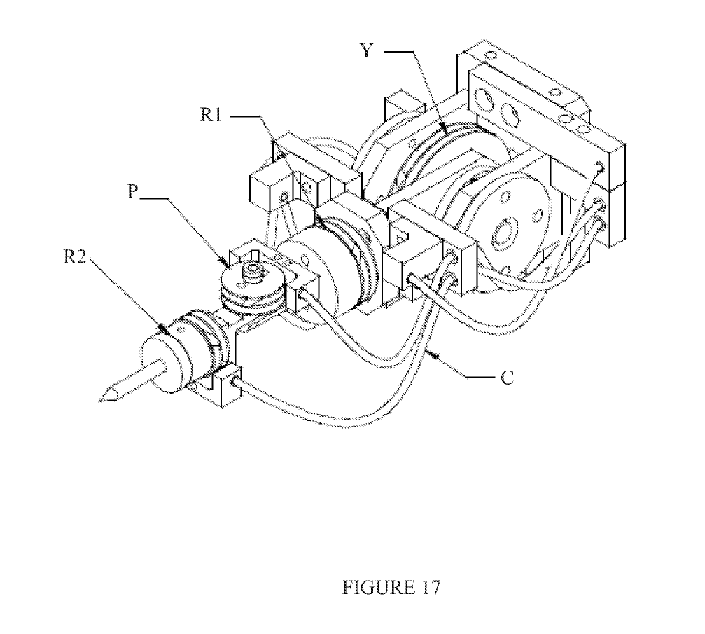

FIG. 17 is an isometric view of an arm wrist and yaw assembly that form part of a surgical arm of the system of FIG. 1. roll 1, roll 2, pitch, yaw and the co-axial driving cables being referenced generally by the alphanumeric characters namely R1, R2, P, Y, and C respectively.

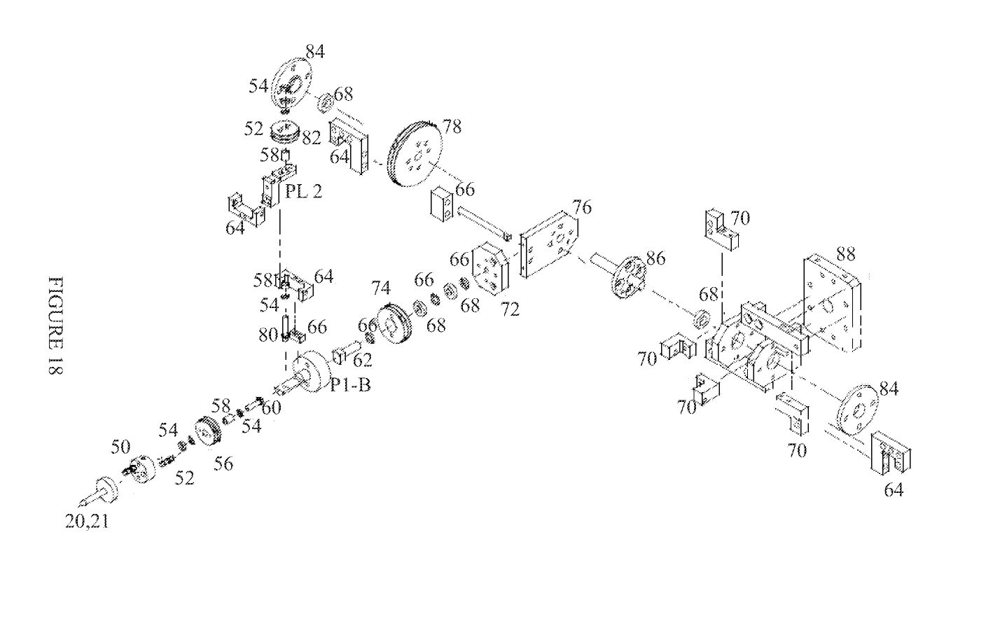

FIG. 18 is an exploded view of FIG. 17 and the key components are referenced generally as follows:
tool 20,21;
tool holder 50
nut 52;
teflon washer 54;
roll 2 pulley 56;
bush 58;
roll 2 shaft 60;
roll 1 shaft 62;
co-axial driving cable mount 64;
pitch base P1-B;
spacer 66;
bearing 68;
co-axial driving cable bracket 70;
roll base 72;
roll 1 pulley 74;
yaw link 76;
yaw pulley 78;
pitch shaft 80;
pitch pulley 82;
bearing cap 84;
yaw shaft 86;
back plate 88; and pitch link P2-L.

The present invention may be embodied in other specific forms without departing from its spirit or essential characteristics. The described embodiment is to be considered in all respects only as illustrative, and not restrictive. The scope of the invention is, therefore, indicated by the appended claims, rather than by the foregoing description. All changes which come within the meaning and range of equivalency of the claims are to be embraced within their scope.

1. A robotic surgical instrument system, said system characterized by:
a plurality of articulating arms having at least two articulation joints, said articulating arms being adapted to be inserted into an operative space in a substantially straight configuration and further adapted to controllably articulate inside the operative space, with at least three degrees of freedom of movement;
at least one access port adapted to receive said articulating arms; and
controlling means adapted to control the articulation of said articulating arms inside the operative space to perform a surgical procedure.

2. The robotic surgical instrument system as claimed in claim 1, wherein said access port is selected from the group consisting of gel ports, puncturable sealed ports and ports with pre-punctured openings.

3. The robotic surgical instrument system as claimed in claim 1, wherein at least two of said articulating arms are surgical arms adapted to hold tools.

4. The robotic surgical instrument system as claimed in claim 1, wherein at least one of said articulating arms is adapted to hold a vision system selected from the group consisting of a fiber optic scope, an insertable camera system and a separate insertable camera.

5. A robotic surgical instrument system, said system characterized by:
a plurality of articulating arms adapted to be inserted into an operative space in a substantially straight configuration and further adapted to controllably articulate inside the operative space, with at least three degrees of freedom of movement;
at least one access port adapted to receive said articulating arms;
controlling means adapted to control the articulation of said articulating arms inside the operative space to perform a surgical procedure; and
at least one vision system adapted to be inserted into said operative space, said vision system being selected from the group consisting of a fiber optic scope, an insertable camera system and a separate insertable camera

6. The robotic surgical instrument system as claimed in claim 1, wherein said controlling means comprises:
at least two external articulated mounting robots co-operating with said articulating arms, said mounting robots having six degrees of freedom and adapted to be floor mounted or ceiling mounted; and
a surgical console adapted to provide an interface for the surgical procedure by a surgeon.

7. The robotic surgical instrument system as claimed in claim 1, wherein said controlling means is adapted to attach tools to or detach tools from said articulating arms.

8. The robotic surgical instrument system as claimed in claim 1, wherein the movement of the articulating arms is achieved by a mechanism comprising cables, pulleys and linkages.

9. A method for a robotic surgical system to access an operative space, said method comprising the following steps:
making an incision in a patient's body;
mounting an access port on the incision;
inserting a plurality of articulating arms into an operative space via the access port in a substantially straight configuration;
controlling said articulating arms inside the operative space to reach a pre-determined operation site by triangulation; and
attaching tools to or detaching tools from said articulating arms.

10. The method for a robotic surgical system to access an operative space as claimed in claim 9, wherein the step of inserting includes a step of inserting at least two articulating arms holding tools and at least one vision system.
